(19) United States
(12) Patent Application Publication
(10) Pub. No.: US 2023/0322796 A1
(43) Pub. Date: Oct. 12, 2023

(54) PROCESS FOR THE PREPARATION OF 2,2-DIFLUORO-1,3-BENZODIOXOLE DERIVATIVES WITH SULFUR CONTAINING SUBSTITUENTS

(71) Applicant: SYNGENTA CROP PROTECTION AG, Basel (CH)

(72) Inventor: Michel MUEHLEBACH, Stein (CH)

(73) Assignee: SYNGENTA CROP PROTECTION AG, Basel (CH)

(21) Appl. No.: 18/043,087

(22) PCT Filed: Aug. 26, 2021
(86) PCT No.: PCT/EP2021/073679

§ 371 Date: Feb. 27, 2023

(30) Foreign Application Priority Data
Aug. 26, 2020 (EP) 20192967.6
Oct. 15, 2020 (EP) 20202088.9

(51) Int. Cl. C07D 491/056; C07D 405/12

(52) U.S. Cl. CPC C07D 491/056; C07D 405/12

(57) ABSTRACT

A process for the preparation of compound of formula (I) is provided, re) wherein X, R₁ and R₂ are as defined in the claim 1.

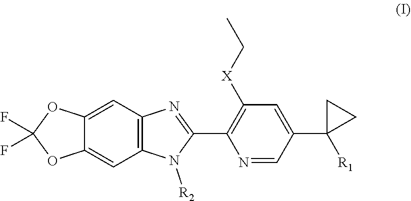

The present invention relates to the preparation of 2,2-difluoro-1,3-benzodioxole derivatives with sulfur containing substituents that are useful as intermediates for the preparation of agrochemicals.

Certain 2,2-difluoro-1,3-benzodioxole derivatives with sulfur containing substituents are useful intermediates for the preparation of biologically active compounds in the agrochemical industries as previously described, for example, in WO 2020/013147, WO 2018/108726, WO 2019/234158, WO 2016/096584 and EP 3 604 300.

In a first aspect, the present invention relates to a process for preparation of 2,2-difluoro-1,3-benzodioxole derivatives with sulfur containing substituents of formula (I)

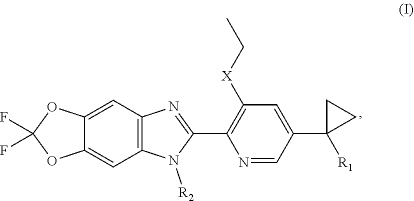

wherein X is S, SO or SO₂; R₁ is H or CN; and R₂ is H or C₁-C₄alkyl, and agrochemically acceptable salts, stereoisomers, enantiomers, tautomers and/or N-oxides of formula (I) comprising a defined number of steps.

In a further aspect, the present invention relates to 2,2-difluoro-1,3-benzodioxole derivatives with sulfur containing substituents of formula (I-1)

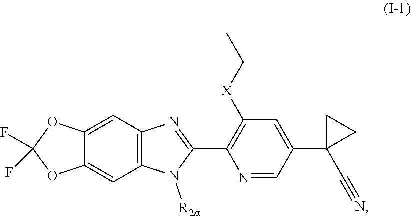

and the agrochemically acceptable salts, stereoisomers, enantiomers, tautomers and N-oxides of formula (I-1), wherein X is S, SO or SO₂; and R₂ₐ is H.

This invention also relates to 2,2-difluoro-1,3-benzodioxole derivatives with sulfur containing substituents of formula (IV) and to a process for preparation thereof.

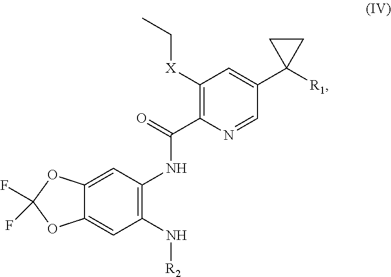

or a salt, stereoisomer, enantiomer, tautomer and/or N-oxide of formula (IV), wherein X is S, SO or SO₂; R₁ is H or CN; and R₂ is H or C₁-C₄alkyl.

The term “C₁-C₄alkyl” as used herein refers to a saturated straight-chain or branched hydrocarbon radical attached via any of the carbon atoms having 1 to 4 carbon atoms, for example, any one of the radicals methyl, ethyl, n-propyl, butyl, sec-butyl, t-butyl.

The preparatory examples within WO 2020/013147 outlines the following reaction:

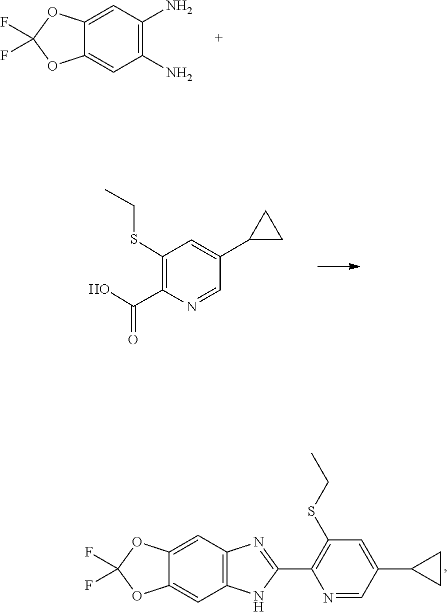 [SMILES: Nc1cc2c(cc1N)OC(F)(F)O2 ; CCSc1cc(C2CC2)cnc1C(=O)O ; CCSc1cc(C2CC2)cnc1-c1nc2cc3c(cc2[nH]1)OC(F)(F)O3]

yet no structural evidence of involved intermediates is disclosed therein. An advantageous route has been found for such compounds. Further, in view of a more efficient and economical route to these useful compounds, it might be advantageous to isolate, purify and characterize occurring intermediates in said process.

Thus, according to the present invention, there is provided a process for the preparation of compound of formula (I)

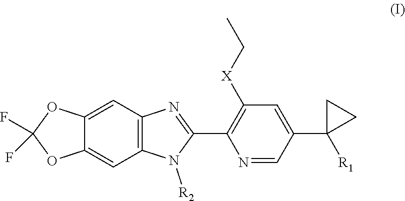

and an agrochemically acceptable salt, stereoisomer, enantiomer, tautomers and/or N-oxide of formula (I), wherein X is S, SO or SO₂; R₁ is H or CN; and R₂ is H or C₁-C₄alkyl;
which process comprises:
(A) reacting a compound of formula (II)

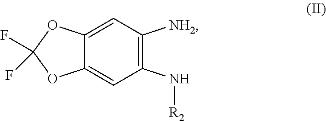

or a salt thereof, wherein R₂ is H or C₁-C₄alkyl;
with a compound of formula (III)

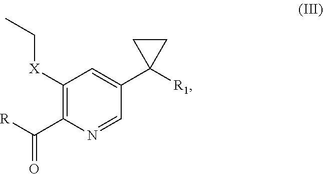

wherein X is S, SO or SO₂; R₁ is H or CN; and R is OH or halogen, preferably chlorine;
optionally in the presence of an activating agent, optionally in the presence of a suitable base, optionally in the presence of an acylation catalyst, in an appropriate solvent (or diluent);
to produce a compound of formula (IV)

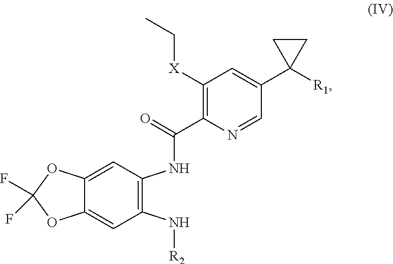

or a salt, stereoisomer, enantiomer, tautomer and/or N-oxide of formula (IV), wherein X is S, SO or SO₂; R₁ is H or CN; and R₂ is H or C₁-C₄alkyl; and
(B) cyclizing a compound of formula (IV)

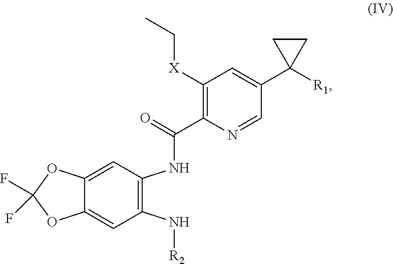

or a salt, stereoisomer, enantiomer, tautomer and/or N-oxide of formula (IV), wherein X is S, SO or SO₂; R₁ is H or CN; and R₂ is H or C₁-C₄alkyl;
in the presence of an acid or an acid catalyst, in an appropriate solvent (or diluent);
to produce the compound of formula (I),

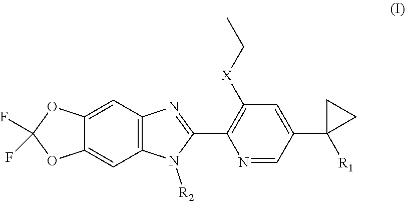

or an agrochemically acceptable salt, stereoisomer, enantiomer, tautomers and/or N-oxide of formula (I), wherein X is S, SO or SO₂; R₁ is H or CN; and R₂ is H or C₁-C₄alkyl.

In a further aspect the present invention provides a compound of formula (IV), or an agrochemically acceptable salt thereof

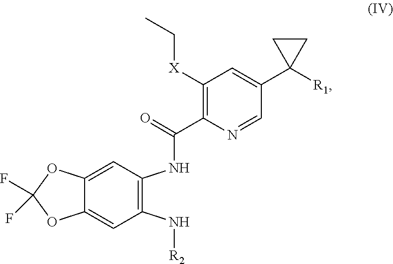

or a regioisomer thereof, wherein X is S, SO or SO₂; R₁ is H or CN; and R₂ is H or C₁-C₄alkyl.

In the particular instance wherein R₂ is C₁-C₄alkyl, the compounds of formula (IV) may exist in form of a regioisomer (IV-1)

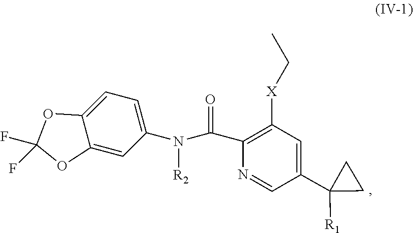

wherein above substituent definitions apply. The present invention also relates to compounds of formulae (IV-1), to a process for preparation of compounds of formulae (IV) and (IV-1) (step (A) above), and to a process for utilization as a reactant thereof (step (B) above), covering both regioisomers, in either pure form, or in a mixture thereof in any ratio.

In yet further aspect of the present invention, a compound of formula (I), represented by a compound of formula (I-1), or an agrochemically acceptable salt thereof, is provided

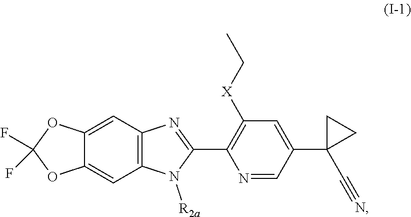

wherein X is S, SO or SO₂; and R₂ₐ is H.

The embodiments described below are applicable to each of, as appropriate, compounds of formulae (I), (II); and (III).
In one embodiment, X is S, SO or SO₂, preferably S or SO₂, even more preferably X is S; R₁ is H or CN; and R₂ is H or C₁-C₄alkyl, preferably H or methyl.
In another embodiment, X is S; R₁ is H or CN; and R₂ is H or C₁-C₄alkyl, preferably H or methyl.
In yet another embodiment, X is SO₂; R₁ is H or CN; and R₂ is H or C₁-C₄alkyl, preferably H or methyl.
In a further embodiment, X is S; R₁ is H; and R₂ is H or C₁-C₄alkyl, preferably H or methyl.
In yet a further embodiment, X is S; R₁ is CN; and R₂ is H or C₁-C₄alkyl, preferably H or methyl.
In a preferred embodiment, X is S; R₁ is H; and R₂ is H or methyl.
In another preferred embodiment, X is S; R₁ is CN; and R₂ is H or methyl.
In a further embodiment, X is SO₂; R₁ is H; and R₂ is H or C₁-C₄alkyl, preferably H or methyl.
In yet a further embodiment, X is SO₂; R₁ is CN; and R₂ is H or C₁-C₄alkyl, preferably H or methyl.

In a preferred embodiment, X is SO₂; R₁ is H; and R₂ is H or methyl.
In another preferred embodiment, X is SO₂; R₁ is CN; and R₂ is H or methyl.

One preferred group of compounds according to this embodiment are compounds of formula (IVa) which are compounds of formula (IV) or (IV-1) wherein X is S, SO or SO₂, preferably S or SO₂, even more preferably X is S; R₁ is H or CN; and R₂ is H or C₁-C₄alkyl, preferably H or methyl.

Another preferred group of compounds according to this embodiment are compounds of formula (IVb) which are compounds of formula (IV) or (IV-1) wherein X is S; R₁ is H or CN; and R₂ is H or C₁-C₄alkyl, preferably H or methyl.

Another preferred group of compounds according to this embodiment are compounds of formula (IVc) which are compounds of formula (IV) or (IV-1) wherein X is SO₂; R₁ is H or CN; and R₂ is H or C₁-C₄alkyl, preferably H or methyl.

Another preferred group of compounds according to this embodiment are compounds of formula (IVd) which are compounds of formula (IV) or (IV-1) wherein X is S; R₁ is H; and R₂ is H or C₁-C₄alkyl, preferably H or methyl.

Another preferred group of compounds according to this embodiment are compounds of formula (IVe) which are compounds of formula (IV) or (IV-1) wherein X is S; R₁ is CN; and R₂ is H or C₁-C₄alkyl, preferably H or methyl.

Another preferred group of compounds according to this embodiment are compounds of formula (IVf) which are compounds of formula (IV) or (IV-1) wherein X is S; R₁ is H; and R₂ is H or methyl.

Another preferred group of compounds according to this embodiment are compounds of formula (IVg) which are compounds of formula (IV) or (IV-1) wherein X is S; R₁ is CN; and R₂ is H or methyl.

Another preferred group of compounds according to this embodiment are compounds of formula (IVh) which are compounds of formula (IV) or (IV-1) wherein X is SO₂; R₁ is H; and R₂ is H or C₁-C₄alkyl, preferably H or methyl.

Another preferred group of compounds according to this embodiment are compounds of formula (IVi) which are compounds of formula (IV) or (IV-1) wherein X is SO₂; R₁ is CN; and R₂ is H or C₁-C₄alkyl, preferably H or methyl.

Another preferred group of compounds according to this embodiment are compounds of formula (IVj) which are compounds of formula (IV) or (IV-1) wherein X is SO₂; R₁ is H; and R₂ is H or methyl.

Another preferred group of compounds according to this embodiment are compounds of formula (IVh) which are compounds of formula (IV) or (IV-1) wherein X is SO₂; R₁ is CN; and R₂ is H or methyl.

One preferred group of compounds according to this embodiment are compounds of formula (I-1a) which are compounds of formula (I-1) wherein X is S, SO or SO₂, preferably S or SO₂; and R₂ₐ is H.

Another preferred group of compounds according to this embodiment are compounds of formula (I-1b) which are compounds of formula (I-1) wherein X is S; and R₂ₐ is H.

Another preferred group of compounds according to this embodiment are compounds of formula (I-1c) which are compounds of formula (I-1) wherein X is SO₂; and R₂ₐ is H.

Compounds of formula (I), wherein X is S, SO or SO₂; R₁ is H or CN; and R₂ is H or C₁-C₄alkyl;

compounds of formula (I-1), wherein X is S, SO or SO₂; and R₂ₐ is H;

compounds of formula (IV), wherein X is S, SO or SO₂; R₁ is H or CN; and R₂ is H or C₁-C₄alkyl; and

compounds of formula (IV-1), wherein X is S, SO or SO₂; R₁ is H or CN; and R₂ is H or C₁-C₄alkyl;

whereby (IV) and (IV-1) may be present in either pure form, or in a mixture thereof in any ratio, may be prepared, isolated, purified and characterized in form of an agrochemically acceptable salt, for example as a hydrohalide salt, preferably a hydrochloride or a hydrobromide salt, or any other equivalent salt.

Compounds of formula (II), or a salt thereof, wherein R₂ is H or C₁-C₄alkyl, are either known, commercially available or may be prepared by methods described in, for example, EP 166287; Journal of Medicinal Chemistry, 2014, 57(19), 7933-7946; Bioorganic & Medicinal Chemistry Letters, 2018, 28(13), 2234-2238; or WO 2020/013147.

Compounds of formula (III), wherein X is S, SO or SO₂; R₁ is H or CN; and R is OH or CI, are either known or may be prepared by methods described in, for example, WO 2016121997, JP 2018012664, WO 2016026848, WO 2018077565 or WO 2016087265.

More generally, compounds of formula (III), wherein X is S, SO or SO₂; R₁ is H or CN; and R is halogen, preferably chlorine, can be prepared from compounds of formula (III), wherein X is S, SO or SO₂; R₁ is H or CN; and R is OH, by activation methods known to those skilled in the art and described in, for example, Tetrahedron, 2005, 61 (46), 10827-10852. For example, compounds (III), wherein R is halogen, preferably chlorine, are formed by treatment of compounds (III), wherein R is OH, with, amongst others, oxalyl chloride (COCl)₂ or thionyl chloride SOCl₂, in the presence of catalytic quantities of N,N-dimethylformamide DMF, in inert solvents such as methylene chloride CH₂Cl₂ or tetrahydrofuran THF, at temperatures between 20 to 100° C., preferably 25° C.

Related to a process for the preparation of compounds of formula (IV), step (A) above, examples of suitable and preferred bases, suitable and preferred activating agents, suitable and preferred acylation catalysts, as well as examples of suitable and preferred reaction conditions (such as solvent (or diluent) and temperature), are given below.

In one embodiment, step (A) comprises

(A-1) reacting a compound of formula (II), or a salt thereof, wherein R₂ is H or C₁-C₄alkyl; with a compound of formula (III), wherein X is S, SO or SO₂; R₁ is H or CN; and R is OH, in the presence of an activating agent, optionally in the presence of a suitable base, in an appropriate solvent (or diluent).

In another embodiment, step (A) comprises

(A-2) reacting a compound of formula (II), or a salt thereof, wherein R₂ is H or C₁-C₄alkyl; with a compound of formula (III), wherein X is S, SO or SO₂; R₁ is H or CN; and R is halogen, preferably chlorine, optionally in the presence of a suitable base, in an appropriate solvent (or diluent).

In a preferred embodiment, step (A) comprises

(A-3) reacting a compound of formula (II), or a salt thereof, wherein R₂ is H or C₁-C₄alkyl; with a compound of formula (III), wherein X is S, SO or SO₂; R₁ is H or CN; and R is OH, in the presence of an activating agent, in the presence of a suitable base, in an appropriate solvent (or diluent).

In a further preferred embodiment, step (A) comprises

(A-4) reacting a compound of formula (II), or a salt thereof, wherein R₂ is H or C₁-C₄alkyl; with a compound of formula (III), wherein X is S, SO or SO₂; R₁ is H or CN; and R is halogen, preferably chlorine, in the presence of a suitable base, in an appropriate solvent (or diluent).

In a further preferred embodiment, step (A) comprises

(A-5) reacting a compound of formula (II), or a salt thereof, wherein R₂ is H or C₁-C₄alkyl; with a compound of formula (III), wherein X is S, SO or SO₂; R₁ is H or CN; and R is halogen, preferably chlorine, in the presence of a suitable base, in the presence of an acylation catalyst, in an appropriate solvent (or diluent).

In a further preferred embodiment, step (A) comprises

(A-6) reacting a compound of formula (II), or a salt thereof, wherein R₂ is H or C₁-C₄alkyl; with a compound of formula (III), wherein X is S, SO or SO₂; R₁ is H or CN; and R is halogen, preferably chlorine, in the presence of a suitable that can also be used as solvent (or diluent).

In a further preferred embodiment, step (A) comprises

(A-7) reacting a compound of formula (II), or a salt thereof, wherein R₂ is H or C₁-C₄alkyl; with a compound of formula (III), wherein X is S, SO or SO₂; R₁ is H or CN; and R is halogen, preferably chlorine, in the presence of an acylation catalyst, in the presence of a suitable that can also be used as solvent (or diluent).

Example of suitable and preferred activating agents for steps (A-1) and (A-3) are amongst useful reagents that activate the carboxylic acid partner for subsequent reaction with amines in amide bond formation, such as propanephosphonic acid anhydride (T3P), carbodiimides (such as dicyclohexylcarbodiimide (DCC), diisopropylcarbodiimide (DIC) and 1-ethyl-3-(3-dimethylamino-propyl)carbodiimide (EDC)), carbodiimides in the presence of ‘racemization suppressing’ additives (such as the triazoles 1-hydroxy-benzotriazole (HOBt), and 1-hydroxy-7-aza-benzotriazole (HOAt)), or aminium/uronium and phosphonium salts (such as HATU (HOAt), HBTU/TBTU (HOBt) and HCTU (6-ClHOBt), and PyBOP (HOBt) and PyAOP (HOAt)). Preferably the activating agent is propanephosphonic acid anhydride or a carbodiimide, even more preferably propanephosphonic acid anhydride.

Example of suitable and preferred bases for steps (A-1), (A-2), (A-3), (A-4), (A-5), (A-6) and (A-7) are triethylamine, diisopropylethylamine, tri-n-propylamine, triethylenediamine, cyclohexylamine, N-cyclohexyl-N,N-dimethylamine, N,N-diethylaniline, quinuclidine, N-methylmorpholine and 1,8-diazabicyclo[5. 4.0]undec-7-ene (DBU), or any mixture thereof. Preferably the base is triethylamine, diisopropylethylamine, pyridine, N-methylmorpholine or N,N-diethylaniline, even more preferably triethylamine, diisopropylethylamine or pyridine. If the reaction is carried out in the presence of a base, for example bases such as triethylamine, diisopropylethylamine, pyridine, N-methylmorpholine or N,N-diethylaniline, can also act as solvents (or diluents).

Example of suitable and preferred acylation catalyst for steps (A-5) and (A-7) is 4-dimethylamino-pyridine (DMAP).

In one embodiment related to the process according to the invention of making compounds of formula (I), step (A), examples of appropriate solvents (or diluents) are dichloromethane, tetrahydrofuran, 2-methyltetrahydrofuran, dioxane, N,N-dimethylformamide, N,N-dimethylacetamide, N-methyl-pyrrolidone, acetonitrile, ethyl acetate, toluene, xylene or chlorobenzene and any mixtures thereof.

In another embodiment, solvent (or diluent) preferred for step (A-6) and (A-7) are triethylamine, diisopropylethylamine, pyridine, N-methylmorpholine or N,N-diethylaniline. Preferably the base is triethylamine, diisopropylethylamine or pyridine.

In one embodiment related to the process according to the invention of making compounds of formula (I), step (A), the reaction is advantageously carried out in a temperature range from approximately 0° C. to approximately 100° C., preferably from approximately 0° C. to approximately 80° C., in many cases in the range between 0° C. and 30° C. In a preferred embodiment, the reaction is carried out in the range between 0° C. and 25° C., such as 5° C. to 25° C.

Related to a process for the preparation of compounds of formula (I), step (B) above, examples of suitable and preferred acids or acid catalysts, as well as examples of suitable and preferred reaction conditions (such as solvent (or diluent) and temperature), are given below.

In one embodiment, step (B) comprises

(B-1) cyclizing a compound of formula (IV), or a salt thereof, or a regioisomer thereof, wherein X is S, SO or SO₂; R₁ is H or CN; and R₂ is H or C₁-C₄alkyl, in the presence of an acid, in an appropriate solvent (or diluent).

In another embodiment, step (B) comprises

(B-2) cyclizing a compound of formula (IV), or a salt thereof, or a regioisomer thereof, wherein X is S, SO or SO₂; R₁ is H or CN; and R₂ is H or C₁-C₄alkyl, in the presence of an acid catalyst, in an appropriate solvent (or diluent).

In a preferred embodiment, step (B) comprises

(B-3) cyclizing a compound of formula (IV), or a salt thereof, or a regioisomer thereof, wherein X is S, SO or SO₂; R₁ is H or CN; and R₂ is H or C₁-C₄alkyl, in in the presence of an acid that can also be used as a solvent (or diluent).

Example of suitable and preferred acids for steps (B-1) and (B-3) are aliphatic acids, such as acetic acid, propionic acid or trifluoroacetic acid. Preferably the acid is acetic acid, even more preferably glacial acetic acid. If the reaction is carried out in the presence of an acid, for example acids such as acetic acid or propionic acid, can also act as solvents (or diluents).

Example of suitable and preferred acid catalysts for step (B-2) are mineral acids, such as hydrochloric acid, sulfuric acid or polyphosphoric acid, sulfonic acids, such as methanesulfonic acid, benzenesulfonic acid or para-toluenesulfonic acid, or dehydrating agents, such as phosphorus pentoxide or acetic anhydride. Preferably the acid catalyst is an arylsulfonic acid, more preferably para-toluene sulfonic acid, even more preferably para-toluene sulfonic acid monohydrate.

In one embodiment related to the process according to the invention of making compounds of formula (I), step (B), examples of appropriate solvents (or diluents) are toluene, xylene, chlorobenzene, N,N-dimethylfomamide, N,N-dimethylacetamide or N-methylpyrrolidone and any mixtures thereof.

In another embodiment, solvent (or diluent) preferred for step (B-2) are toluene or N,N-dimethylfomamide and any mixtures thereof, more preferably a mixture of toluene and N,N-dimethylfomamide in a 4:1 ratio.

In another embodiment, the acid that can also be used as a solvent for step (B-3) are acetic acid, propionic acid or trifluoroacetic acid. Preferably the acid is acetic acid, even more preferably glacial acetic acid.

In one embodiment related to the process according to the invention of making compounds of formula (I), step (B), the reaction is advantageously carried out in a temperature range from approximately 25° C. to approximately 180° C., preferably from approximately 80° C. to approximately 170° C., in many cases in the range between 100° C. and up to the boiling point of the reaction mixture.

Compounds of formulae (I) and (I-1), which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula I which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

In each case, the compounds of formulae (I) and (I-1), according to the invention are in free form, in oxidized form as a N-oxide or in salt form, e.g. an agronomically usable salt form. N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The compounds of formulae (I) and (I-1), according to the invention, also include hydrates which may be formed during the salt formation.

Compounds of formula (I-1) are useful insecticides and can be formulated and mixed with other active ingredients to expand its biological spectrum/potency to control damage by pests in plants and other fields.

### PREPARATORY EXAMPLES

“Mp” means melting point in ° C. Free radicals represent methyl groups. ¹H NMR measurements were recorded on a Brucker 400 MHz spectrometer, chemical shifts are given in ppm relevant to a TMS standard. Spectra measured in deuterated solvents as indicated. The LCMS method below was used to characterize the compounds. The characteristic LCMS values obtained for each compound were the retention time (“Rt”, recorded in minutes) and the measured molecular ion (M+H)⁺ or (M−H)⁻.

### LCMS Methods:

### Method 1

Spectra were recorded on a Mass Spectrometer from Waters Corporation (SQD, SQDII or QDA Single quadrupole mass spectrometer) equipped with an electrospray source (Polarity: positive and negative ions), Capillary: 0.8-3.00 kV, Cone: 5-30 V, Source Temperature: 120-150° C., Desolvation Temperature: 350-600° C., Cone Gas Flow: 50-150 l/h, Desolvation Gas Flow: 650-1000 l/h, Mass range: 50 to 900 Da and an Acquity UPLC from Waters Corporation: Binary pump, heated column compartment , diode-array detector and ELSD. Column: Waters UPLC HSS T3, 1.8 μm, 30×2.1 mm, Temp: 60° C., DAD Wavelength range (nm): 210 to 400, Runtime: 1.5 min; Solvents: A=water+5% MeOH+0.05% HCOOH, B=Acetonitrile+0.05% HCOOH; Flow (ml/min) 0.85, Gradient: 10% B isocratic for 0.2 min, then 10-100% B in 1.0 min, 100% B isocratic for 0.2 min, 100-10% B in 0.05 min, 10% B isocratic for 0.05 min.

### Example 1: Preparation of 5-(1-cyanocyclopropyl)-N-[2,2-difluoro-6-(methylamino)-1,3-benzodioxol-5-yl]1-3-ethylsulfanyl-pyridine-2-carboxamide

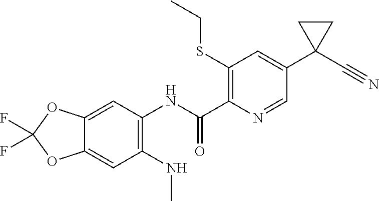 [SMILES: CCSc1cc(C2(C#N)CC2)cnc1C(=O)Nc1cc2c(cc1NC)OC(F)(F)O2]

To a solution of 2,2-difluoro-N5-methyl-1,3-benzodioxole-5,6-diamine hydrochloric salt (69 mg, 0.29 mmol) in tetrahydrofuran (0.97 mL) at room temperature were added 4-dimethylamino-pyridine (0.3 mg, 0.01 eq.), triethylamine (0.1 mL, 0.72 mmol), followed by a solution of 5-(1-cyanocyclopropyl)-3-ethylsulfanyl-pyridine-2-carbonyl chloride (64 mg, 0.24 mmol) in tetrahydrofuran (0.97 mL) dropwise. The mixture was stirred at room temperature for one hour. The reaction mixture was diluted with aqueous sodium hydrogen carbonate and ethyl acetate, the product extracted twice with ethyl acetate, the combined organic layers washed with an aqueous saturated solution of sodium hydrogen carbonate, dried over magnesium sulfate, filtered and concentrated in vacuo. The residue was purified by Combiflash (gradient ethyl acetate in cyclohexane) to afford the desired product. LCMS (method 1): retention time 1.11 min, m/z 433 (M+H)⁺. ¹H NMR (400 MHz, CDCl₃) δ ppm 1.46 (t, 3H), 1.57 (m, 2H), 1.93 (m, 2H), 2.85 (br s, 3H), 2.98 (q, 2H), 3.73 (br s, 1H), 6.57 (s, 1H), 7.48 (s, 1H), 7.68 (d, 1H), 8.15 (d, 1H), 9.61 (br s, 1H).

### Example 2: Preparation of 1-[6-(2,2-difluoro-7-methyl-[1,3]dioxolol[4,5-f]benzimidazol-6-yl)-5-ethylsulfanyl-3-pyridyl]cyclopropanecarbonitrile

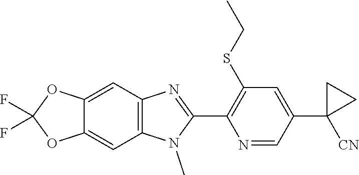 [SMILES: CCSc1cc(C2(C#N)CC2)cnc1-c1nc2cc3c(cc2n1C)OC(F)(F)O3]

A solution of 5-(1-cyanocyclopropyl)-N-[2,2-difluoro-6-(methylamino)-1,3-benzodioxol-5-yl]-3-ethylsulfanyl-pyridine-2-carboxamide (prepared as described in Example 1) (33.9 mg, 0.078 mmol) was refluxed in glacial acetic acid (0.3 mL) for two hours. The mixture was concentrated in vacuo and the residue diluted with ethyl acetate and aqueous sodium hydrogen carbonate. The product was extracted twice with ethyl acetate, the combined organic layers washed with an aqueous saturated solution of sodium hydrogen carbonate, dried over magnesium sulfate, filtered and concentrated in vacuo. The residue was purified by Combiflash (gradient ethyl acetate in cyclohexane) to afford the desired product. LCMS (method 1): retention time 1.05 min, m/z 415 (M+H)⁺. ¹H NMR (400 MHz, CDCl₃) δ ppm 1.36 (t, 3H), 1.56 (m, 2H), 1.89 (m, 2H), 2.97 (q, 2H), 3.89 (s, 3H), 7.09 (s, 1H), 7.52 (s, 1H), 7.69 (d, 1H), 8.31 (d, 1H).

### Example 3: Preparation of N-(6-amino-2,2-difluoro-1,3-benzodioxol-5-yl)-5-(1-cyanocyclopropv1)-3-ethylsulfanyl-pyridine-2-carboxamide

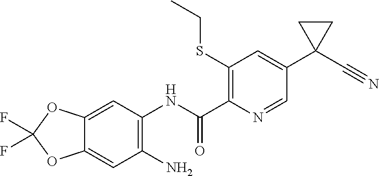 [SMILES: CCSc1cc(C2(C#N)CC2)cnc1C(=O)Nc1cc2c(cc1N)OC(F)(F)O2]

To a solution of 2,2-difluoro-1,3-benzodioxole-5,6-diamine (78.4 mg, 0.40 mmol) in tetrahydrofuran (1.6 mL) at room temperature were added 4-dimethylamino-pyridine (0.49 mg, 0.01 eq.), triethylamine (0.11 mL, 0.80 mmol), followed by a solution of 5-(1-cyanocyclopropyl)-3-ethylsulfanyl-pyridine-2-carbonyl chloride (107 mg, 0.40 mmol) in tetrahydrofuran (1.6 mL) dropwise. The mixture was stirred at room temperature for one hour. The reaction mixture was diluted with aqueous sodium hydrogen carbonate and ethyl acetate, the product extracted twice with ethyl acetate, the combined organic layers washed with an aqueous saturated solution of sodium hydrogen carbonate, dried over magnesium sulfate, filtered and concentrated in vacuo. The residue was purified twice by Combiflash (gradient ethyl acetate in cyclohexane, then tert-butyl methyl ether in cyclohexane) to afford the desired product. LCMS (method 1): retention time 1.05 min, m/z 419 (M+H)⁺. ¹H NMR (400 MHz, CDCl₃) δ ppm 1.46 (t, 3H), 1.57 (m, 2H), 1.92 (m, 2H), 2.98 (q, 2H), 3.67 (br s, 2H), 6.60 (s, 1H), 7.51 (s, 1H), 7.68 (d, 1H), 8.14 (d, 1H), 9.76 (s, 1H).

### Example 4: Preparation of 1-[6-(2,2-difluoro-5H-[1,3]dioxolol[4,5-f]benzimidazol-6-yl)-5-ethylsulfanyl-3-pyridyl]cyclopropanecarbonitrile

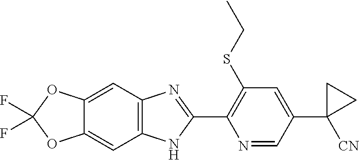 [SMILES: CCSc1cc(C2(C#N)CC2)cnc1-c1nc2cc3c(cc2[nH]1)OC(F)(F)O3]

A solution of N-(6-amino-2,2-difluoro-1,3-benzodioxol-5-yl)-5-(1-cyanocyclopropyl)-3-ethylsulfanyl-pyridine-2-carboxamide (prepared as described in Example 3) (65 mg, 0.16 mmol) in glacial acetic acid (0.89 mL) was heated to 110° C. for 3 hours. The mixture was added to iced water and the product extracted with ethyl acetate. The combined organic layers were washed with water and brine, dried over sodium sulfate, filtered and concentrated in vacuo. The residue was purified by Combiflash (gradient ethyl acetate in cyclohexane) to afford the desired product. LCMS (method 1): retention time 1.08 min, m/z 399 (M−H)⁻. ¹H NMR (400 MHz, CDCl₃) δ ppm 1.50 (t, 3H), 1.57 (m, 2H), 1.91 (m, 2H), 3.09 (q, 2H), 7.47 (br s, 2H), 7.69 (d, 1H), 8.21 (d, 1H).

### Example 5: Preparation of N-(6-amino-2,2-difluoro-1,3-benzodioxol-5-yl)-5-cyclopropyl-3-ethylsulfanyl-pyridine-2-carboxamide

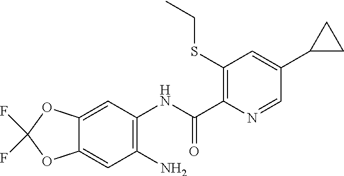 [SMILES: CCSc1cc(C2CC2)cnc1C(=O)Nc1cc2c(cc1N)OC(F)(F)O2]

To a solution of 2,2-difluoro-1,3-benzodioxole-5,6-diamine (78.4 mg, 0.40 mmol) in tetrahydrofuran (1.6 mL) at room temperature were added 4-dimethylamino-pyridine (0.49 mg, 0.01 eq.), triethylamine (0.11 mL, 0.80 mmol), followed by a solution of 5-cyclopropyl-3-ethylsulfanyl-pyridine-2-carbonyl chloride (96.7 mg, 0.40 mmol) in tetrahydrofuran (1.6 mL) dropwise. The mixture was stirred at room temperature for one hour. The reaction mixture was slowly diluted with aqueous sodium hydrogen carbonate and ethyl acetate, the product extracted twice with ethyl acetate, the combined organic layers washed with an aqueous saturated solution of sodium hydrogen carbonate, dried over magnesium sulfate, filtered and concentrated in vacuo. The residue was purified by Combiflash (gradient ethyl acetate in cyclohexane) to afford the desired product. LCMS (method 1): retention time 1.12 min, m/z 394 (M+H)⁺. ¹H NMR (400 MHz, CDCl₃) δ ppm 0.84 (m, 2H), 1.16 (m, 2H), 1.43 (t, 3H), 1.98 (m, 1H), 2.93 (q, 2H), 3.70 (br s, 2H), 6.59 (s, 1H), 7.31 (d, 1H), 7.49 (s, 1H), 8.06 (d, 1H), 9.80 (s, 1H).

### Example 6: Preparation of 6-(5-cyclopropyl-3-ethylsulfanyl-2-pyridyl)-2,2-difluoro-5H-[1,3]dioxolo[4,5-f]benzimidazole

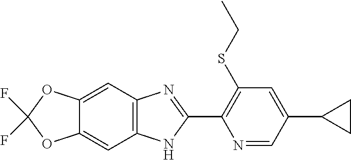 [SMILES: CCSc1cc(C2CC2)cnc1-c1nc2cc3c(cc2[nH]1)OC(F)(F)O3]

To a solution N-(6-amino-2,2-difluoro-1,3-benzodioxo1-5-yl)-5-cyclopropyl-3-ethylsulfanyl-pyridine-2-carboxamide (prepared as described in Example 5) (58.6 mg, 0.15 mmol) in a mixture of toluene (1.86 mL) and N,N-dimethylformamide (0.46 mL) was added p-toluenesulfonic acid hydrate (28.3 mg, 0.15 mmol). The reaction mixture was heated in the microwave at 150° C. for 40 minutes, then poured into iced water. The product was extracted with ethyl acetate, the combined organic layers washed with water and brine, dried over sodium sulfate, filtered and concentrated in vacuo. The residue was purified by Combiflash (gradient ethyl acetate in cyclohexane) to afford the desired product. LCMS (method 1): retention time 1.14 min, m/z 374 (M−H)⁻. ¹H NMR (400 MHz, CDCl₃) δ ppm 0.83 (m, 2H), 1.14 (m, 2H), 1.47 (t, 3H), 1.96 (m, 1H), 3.04 (q, 2H), 7.35 (d, 1H), 7.37 (br s, 2H), 8.11 (d, 1H).

### Example 7: Preparation of 5-cyclopropyl-N-[2,2-difluoro-6-(methylamino)-1,3-benzodioxol-5-yl]-3-ethylsulfanyl-pyridine-2-carboxamide

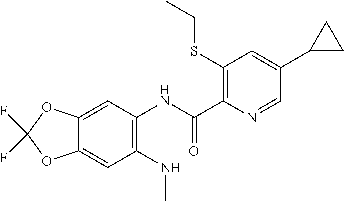 [SMILES: CCSc1cc(C2CC2)cnc1C(=O)Nc1cc2c(cc1NC)OC(F)(F)O2]

To a solution of 5-cyclopropyl-3-ethylsulfanyl-pyridine-2-carboxylic acid (50 mg, 0.22 mmol) in ethyl acetate (0.75 mL) under nitrogen at 0° C. were added 2,2-difluoro-N5-methyl-1,3-benzodioxole-5,6-diamine hydrochloric salt (64.1 mg, 0.27 mmol), N,N-diisopropylethylamine (0.09 mL, 0.52 mmol) and a 50% solution of T3P [propanephosphonic acid anhydride] in methyl-tetrahydrofuran (0.178 mL, 0.29 mmol) dropwise. The mixture was stirred at 0° C. for 2.5 hours, additional T3P solution (0.06 mL) was added and stirring continued at 0° C. for 1 hour. The reaction mixture was diluted with aqueous sodium hydrogen carbonate, the product extracted twice with ethyl acetate, the combined organic layers washed with an aqueous saturated solution of sodium hydrogen carbonate, dried over magnesium sulfate, filtered and concentrated in vacuo. The residue was purified by Combiflash (gradient ethyl acetate in cyclohexane) to afford the desired product. LCMS (method 1): retention time 1.20 min, m/z 408 (M+H)³⁰ . ¹H NMR (400 MHz, CDCl₃) δ ppm 0.82 (m, 2H), 1.15 (m, 2H), 1.41 (t, 3H), 1.95 (m, 1H), 2.82 (s, 3H), 2.91 (q, 2H), 3.35 (br s, 1H), 6.52 (s, 1H), 7.28 (d, 1H), 7.46 (s, 1H), 8.03 (d, 1H), 9.65 (s, 1H).

### Example 8: Preparation of 6-(5-cyclopropyl-3-ethylsulfanyl-2-pyridyl)-2,2-difluoro-7-methyl-[1,3]dioxolo[4,5-f]benzimidazole

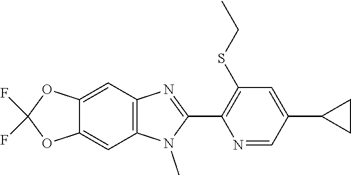 [SMILES: CCSc1cc(C2CC2)cnc1-c1nc2cc3c(cc2n1C)OC(F)(F)O3]

Obtained from 5-cyclopropyl-N-[2,2-difluoro-6-(methylamino)-1,3-benzodioxo1-5-yl]-3-ethylsulfanyl-pyridine-2-carboxamide (prepared as described in Example 7) in glacial acetic acid according to the procedure of Example 2 above. The reaction mixture was refluxed for 2 hours and the crude product obtained after extractive work-up was purified by Combiflash (gradient ethyl acetate in cyclohexane) to afford the desired product. LCMS (method 1): retention time 1.14 min, m/z 390 (M+H)⁺. ₁H NMR (400 MHz, CDCl₃) δ ppm 0.84 (m, 2H), 1.14 (m, 2H), 1.32 (t, 3H), 1.98 (m, 1H), 2.91 (q, 2H), 3.85 (s, 3H), 7.08 (s, 1H), 7.37 (d, 1H), 7.51 (s, 1H), 8.26 (d, 1H).

### Example 9: Preparation of N-(6-amino-2,2-difluoro-1,3-benzodioxo1-5-yl)-5-(1-cyanocyclopropyl)-3-ethylsulfonyl-pyridine-2-carboxamide

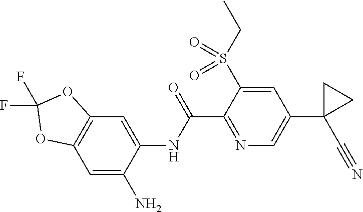 [SMILES: CCS(=O)(=O)c1cc(C2(C#N)CC2)cnc1C(=O)Nc1cc2c(cc1N)OC(F)(F)O2]

To a solution of 2,2-difluoro-1,3-benzodioxole-5,6-diamine (276 mg, 1.41 mmol) in tetrahydrofuran (5.4 mL) at room temperature were added 4-dimethylamino-pyridine (1.64 mg, 0.01 eq.), triethylamine (0.373 mL, 2.68 mmol), followed by a solution of 5-(1-cyanocyclopropyI)-3-ethylsulfonyl-pyridine-2-carbonyl chloride (400 mg, 1.34 mmol) in tetrahydrofuran (5.4 mL) dropwise. The mixture was stirred at room temperature for 16 hours. The reaction mixture was diluted with aqueous sodium hydrogen carbonate and ethyl acetate, the product extracted twice with ethyl acetate, the combined organic layers washed with an aqueous saturated solution of sodium hydrogen carbonate, dried over magnesium sulfate, filtered and concentrated in vacuo. The residue was purified by Combiflash (gradient ethyl acetate in cyclohexane) to afford the desired product. LCMS (method 1): retention time 0.91 min, m/z 451 (M+H)⁺. ¹H NMR (400 MHz, CDCl₃) δ ppm 1.37 (t, 3H), 1.64 (m, 2H), 2.02 (m, 2H), 3.88 (q, 2H), 3.90 (br s, 2H), 6.59 (s, 1H), 7.26 (s, 1H), 8.21 (d, 1H), 8.70 (br s, 1H), 8.92 (d, 1H).

### Example 10: Preparation of 1-[6-(2,2-difluoro-5H-[1,3]dioxolol[4,5-f]benzimidazol-6-yl)-5-ethylsulfonyl-3-pyridyl]cyclopropanecarbonitrile

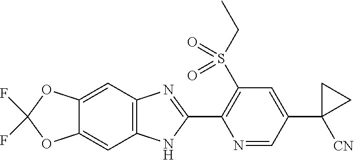 [SMILES: CCS(=O)(=O)c1cc(C2(C#N)CC2)cnc1-c1nc2cc3c(cc2[nH]1)OC(F)(F)O3]

A solution of N-(6-amino-2,2-difluoro-1,3-benzodioxo1-5-yl)-5-(1-cyanocyclopropyI)-3-ethylsulfonyl-pyridine-2-carboxamide (prepared as described in Example 9) (191.4 mg, 0.42 mmol) in glacial acetic acid (1.72 mL) was refluxed for 2 hours. The mixture was concentrated in vacuo, the residue diluted with ethyl acetate and aqueous sodium hydrogen carbonate, the product extracted twice with ethyl acetate, the combined organic layers washed with an aqueous saturated solution of sodium hydrogen carbonate, dried over magnesium sulfate, filtered and concentrated in vacuo. The residue was purified by Combiflash (gradient ethyl acetate in cyclohexane) to afford the desired product as a white solid. LCMS (method 1): retention time 1.00 min, m/z 433 (M+H)⁺. ¹H NMR (400 MHz, d6 DMSO) δ ppm 1.28 (t, 3H), 1.87 (m, 2H), 1.98 (m, 2H), 4.28 (q, 2H), 7.57 (s, 1H), 7.78 (s, 1H), 8.41 (d, 1H), 8.91 (d, 1H), 13.49 (br s, 1H).

### Example 11: Preparation of 5-(1-cyanocyclopropyl)-N-[2,2-difluoro-6-(methylamino)-1,3-benzodioxol-5-yl]-3-ethylsulfonyl-pyridine-2-carboxamide

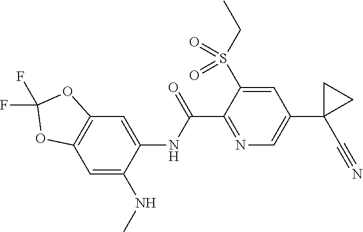 [SMILES: CCS(=O)(=O)c1cc(C2(C#N)CC2)cnc1C(=O)Nc1cc2c(cc1NC)OC(F)(F)O2]

To a solution of 2,2-difluoro-N5-methyl-1,3-benzodioxole-5,6-diamine hydrochloric salt (176 mg, 0.74 mmol) in tetrahydrofuran (2.7 mL) at room temperature were added 4-dimethylamino-pyridine (0.82 mg, 0.01 eq.), triethylamine (0.28 mL, 2.01 mmol), followed by a solution of 5-(1-cyanocyclopropyl)-3-ethylsulfonyl-pyridine-2-carbonyl chloride (200 mg, 0.67 mmol) in tetrahydrofuran (2.7 mL) dropwise. The mixture was stirred at room temperature for 16 hours. The reaction mixture was diluted with aqueous sodium hydrogen carbonate and ethyl acetate, the product extracted twice with ethyl acetate, the combined organic layers washed with an aqueous saturated solution of sodium hydrogen carbonate, dried over magnesium sulfate, filtered and concentrated in vacuo. The residue was purified by Combiflash (gradient ethyl acetate in cyclohexane) to afford the desired product. LCMS (method 1): retention time 0.99 min, m/z 465 (M+H)⁺. ¹H NMR (400 MHz, CDCl₃) δ ppm 1.37 (t, 3H), 1.64 (m, 2H), 2.02 (m, 2H), 2.86 (s, 3H), 3.86 (q, 2H), 4.16 (br s, 1H), 6.55 (s, 1H), 7.25 (s, 1H), 8.20 (d, 1H), 8.44 (br s, 1H), 8.93 (d, 1H).

1. A process for the preparation of a compound of formula (I) 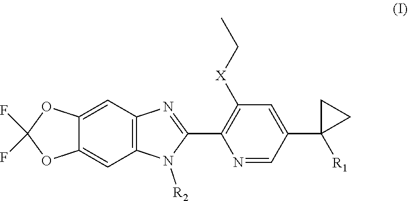
and an agrochemically acceptable salt, stereoisomer, enantiomer, tautomer and/or N-oxide of formula (I), wherein X is S, SO or SO₂; R₁ is H or CN; and R₂ is H or C₁-C₄alkyl;
which process comprises:
(A) reacting a compound of formula (II) 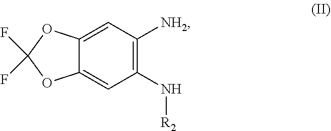
or a salt thereof, wherein R₂ is H or C₁-C₄alkyl;
with a compound of formula (III) 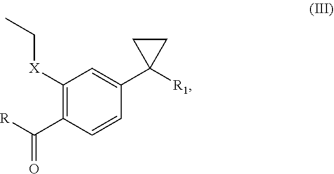
wherein X is S, SO or SO₂; R₁ is H or CN; and R is OH or halogen, in an appropriate solvent (or diluent);
to produce a compound of formula (IV) 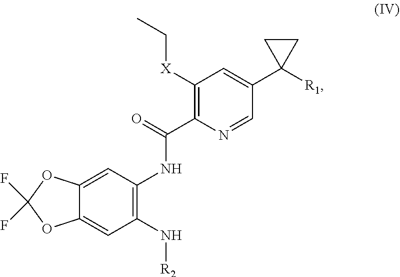
or a salt thereof, or a regioisomer thereof, wherein X is S, SO or SO₂; R₁ is H or CN; and R₂ is H or C₁-C₄alkyl; and
(B) cyclizing a compound of formula (IV) 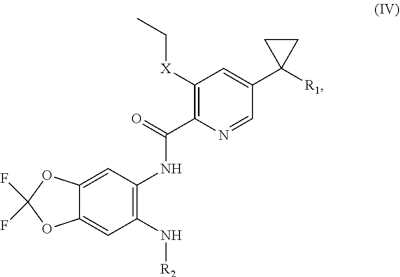
or a salt thereof, or a regioisomer thereof, wherein X is S, SO or SO₂; R₁ is H or CN; and R₂ is H or C₁-C₄alkyl;
in the presence of an acid or an acid catalyst, in an appropriate solvent (or diluent);
to produce the compound of formula (I), 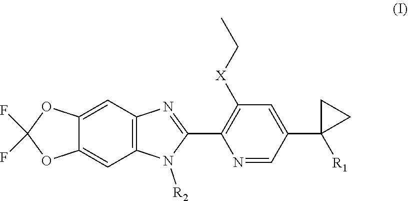
or an agrochemically acceptable salt, stereoisomer, enantiomer, tautomer and/or N-oxide of formula (I-1), wherein X is S, SO or SO₂; R₁ is H or CN; and R₂ is H or C₁-C₄alkyl.

2. The process according to claim 1, wherein a compound of formula (III) wherein R is chlorine is reacted in step (A).

3. The process according to claim 1, wherein in step (A) the compound of formula (II) is reacted with the compound of formula (III) in the presence of an activating agent.

4. The process according to claim 1, wherein in step (A) the compound of formula (II) is reacted with the compound of formula (III) in the presence of a suitable base.

5. The process according to claim 1, wherein in step (A) the compound of formula (II) is reacted with the compound of formula (III) in the presence of an acylation catalyst.

6. The process according to claim 1, wherein X in each of formulae (I), (II), and (IV) is either S or SO₂.

7. The process according to claim 1, wherein R₂ is H or methyl.

8. The process according to claim 1, wherein step (A) is in the presence of at least one of an activating agent, a suitable base, and an appropriate solvent (or diluent).

9. The process according to claim 1, wherein step (A) is in the presence of at least one of an acylation catalyst, a suitable base, and an appropriate solvent (or diluent).

10. The process according to claim 1, wherein in step (B) the acid is selected from acetic acid, propionic acid and trifluoroacetic acid.

11. The process according to claim 1, wherein in step (B) the acid catalyst is selected from hydrochloric acid, sulfuric acid, polyphosphoric acid, methanesulfonic acid, benzenesulfonic acid, para-toluenesulfonic acid, para-toluene sulfonic acid monohydrate, phosphorus pentoxide and acetic anhydride.

12. A compound of formula (IV) 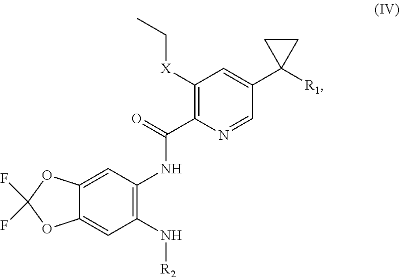
or a salt, stereoisomer, enantiomer, tautomer and/or N-oxide of formula (IV), wherein X is S, SO or SO₂; R₁ is H or CN; and R₂ is H or C₁-C₄alkyl.

13. A compound of formula (I-1) 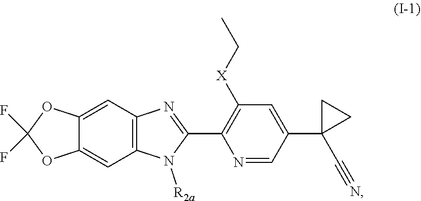
and an agrochemically acceptable salt, stereoisomer, enantiomer, tautomer and/or N-oxide of formula (I-1), wherein X is S, SO or SO₂; and R₂ₐ is H.
